(19) United States
(12) Patent Application Publication
(10) Pub. No.: US 2025/0123274 A1
(43) Pub. Date: Apr. 17, 2025

(54) ALVEOLAR ORGANOIDS, METHODS OF MAKING AND USES THEREOF

(71) Applicants: Versitech Limited, Hong Kong (CN); Centre for Virology, Vaccinology and Therapeutics Limited, Hong Kong (CN)

(72) Inventors: Jie Zhou, Hong Kong (CN); Man Chun Chiu, Hong Kong (CN); Cun Li, Hong Kong (CN); Kwok Yung Yuen, Hong Kong (CN)

(21) Appl. No.: 18/688,178

(22) PCT Filed: Aug. 25, 2022
(86) PCT No.: PCT/CN2022/114792

§ 371 Date: Feb. 29, 2024

Related U.S. Application Data
(60) Provisional application No. 63/238,486, filed on Aug. 30, 2021.

(51) Int. Cl. G01N 33/50; C12N 5/071

(52) U.S. Cl. CPC G01N 33/5091; C12N 5/0688; G01N 33/5082; C12N 2501/415; C12N 2533/90; G01N 2333/165

(57) ABSTRACT

Methods for obtaining a population of differentiated alveolar cells, differentiated alveolar cells in the form of alveolar organoids generated by the methods and uses for the differentiated alveolar organoids are provided. The methods include digesting long-term expanding lung organoids (LO) into single cells (i.e., dissociated cells), and suspension culturing the dissociated cells in distal differentiation (DD) cell culture media in a non-adherent plate for an effective amount of time. Alveolar organoids includes a population of AT1 and AT2 cells and in suspension culture exhibit an apical-out polarity and include abundant cytoplasmic lamellar bodies and microvilli. The alveolar organoids can be utilized alone or in combination with 2D and 3D AWO, rebuilt the human respiratory epithelium in culture plates for studying biology and pathology of human respiratory system, including, but not limited to, the study of COVID-19 respiratory diseases.

### FIELD OF THE INVENTION

The invention is generally directed to alveolar organoids, methods of making and using.

### BACKGROUND OF THE INVENTION

The human respiratory tract is covered with two distinct types of epithelia, the proximal airway epithelium and distal alveolar epithelium (FIG. 1A). The former lines the airway from nasal cavity to terminal bronchiole, consisting of four major types of epithelial cells, i.e. ciliated cell, goblet cell, Club cell and basal cell. Human alveolar sac, the basic unit of ventilation and oxygen exchange, is covered with alveolar epithelium, consisting of flat type I alveolar epithelial (AT1) cell and cuboidal type II alveolar epithelial (AT2) cell. Human respiratory epithelial cells cannot be long-term and stably maintained and expanded in vitro. Currently, immortalized cell lines such as human lung adenocarcinoma cell line A549 or Calu3 have been commonly used for studying respiratory diseases, including respiratory infection of SARS-CoV-2. However, these homogenous cell lines cannot simulate the multicellular complexity and functional diversity of human respiratory epithelia, let alone to model and study human biology and pathology. Primary human alveolar epithelial cells are commercially available. However, they are not expandable and very expensive since they are very likely procured from lung tissues of donated cadaver which hamper the use of these primary for routine research application.

Lack of primary respiratory epithelial cells critically hampers the research of respiratory diseases, including the pandemic COVID-19.

Therefore, it is the object of the present invention to provide methods of making alveolar organoids.

It is another object of the present invention to provide compositions for making alveolar organoids.

It is yet another object of the present invention to provide alveolar organoids derived in vitro.

### SUMMARY OF THE INVENTION

Methods for obtaining a population of differentiated alveolar cells, differentiated alveolar cells generated by the disclosed methods and uses for the differentiated alveolar cells are provided. The population of differentiated alveolar cells are in the form of an organoid, herein alveolar organoid (AlvO).

The methods include digesting long-term expandable lung organoids (LO) into single cells (i.e., dissociated cells), culturing the dissociated cells in distal differentiation (DD) cell culture media, followed by suspension culture in a non-adherent plate with distal differentiation medium for an effective amount of time, preferably, at least two weeks, following which, physiologically-active alveolar organoids are obtained. Long term expandable lung organoids are obtained preferably, from a lung tissue sample from a subject.

Alveolar organoids also disclosed, which includes a population of AT1 and AT2 cells, as evidenced by expression of canonical AT1 cell markers AQP5 (aquaporin 5), HOPX (homeodomain-only protein homeobox) and AT2 cell markers SFTPA (pulmonary-surfactant associated protein A), SFTPB (pulmonary-surfactant associated protein B), SFTPC (pulmonary-surfactant associated protein C), SFTPD (pulmonary-surfactant associated protein D). The alveolar organoids in suspension culture exhibit an apical-out polarity and include abundant cytoplasmic lamellar bodies and microvilli.

The disclosed alveolar organoids can be utilized alone or in combination with 2D and 3D AWO (FIG. 3), to rebuild the human respiratory epithelium in culture plates for studying biology and pathology of human respiratory system, including, but not limited to, the study of COVID-19 respiratory diseases caused by SARS coronavirus 2.

### BRIEF DESCRIPTION OF THE DRAWINGS

FIG. 1A is a cartoon showing the human respiratory epithelium. FIG. 1B is a schematic graph outlines the generation of alveolar organoids. Photomicrographs of live lung organoids (LO) and differentiated alveolar organoids (AlvO) are presented on the left, scale bar, 100 μm. Confocal images of the corresponding organoids (right) labeled with DAPI (blue) and Phalloidin (white), scale bar, 20 μm. FIG. 1C shows LOs and AlvOs differentiated from the same line were assessed for the transcriptional level of AT1- and AT2-cell marker genes. Data represent the mean and s.d. of a representative experiment, n=6. Independent experiments were performed in organoids derived from 3 different donors. Two-tailed Student's t-test. FIG. 1D shows AlvOs subjected to immunofluorescence staining to label AQP5+ AT1 cells, SFTPB+ or HTII-280+AT2 cells. Nuclei and actin filaments were counterstained with DAPI (blue) and Phalloidin-647 (white), respectively. Scale bar, 20 μm. FIG. 1E shows LOs and AlvOs differentiated from the same line were applied to flow cytometry to detect the abundance of AQP5+ AT1 and SFTPC+ AT2 cell. Representative histograms are shown on the top. Quantitative data underneath represent the mean and s.d. of a representative experiment repeated in 3 lines of organoids. n=3. Two-tailed Student's t-test. FIG. 1F-1I. AlvOs were examined with transmission electro-microscopy. FIG. 1F shows cuboidal AT2 cells and thin AT1 cells in an alveolar organoid. Scale bar, 10 μm. FIG. 1G shows microvilli on an AT2 cell. Scale bar, 2 μm. (FIG. 1H) Abundant lamella bodies in an AT2 cell. Scale bar, 1 μm. (FIG. 1G) A lamella body within an AT2 cell. Scale bar, 0.2 μm (i) An AT1 cell with thin cytoplasm. Scale bar, 20 μm. (i) AlvOs pre-incubated with BODIPY phosphatidylcholine were labelled with LysoTracker Red, and then subject to live-cell confocal imaging. Organoids were counterstained with Hoechst (blue) and CellMask™ plasma membrane stain (white). Scale bar, 20 μm. FIG. 1K shows the result of AlvOs subjected to immunofluorescence staining to label HTII-280+ AT2 cells and ACE2 expressing cells. Nuclei and actin filaments were counterstained with DAPI (blue) and Phalloidin-647 (white), respectively. Scale bar, 20 μm.

FIG. 2A-2C show SARS-CoV-2 infection in alveolar organoids infection. (FIG. 2A) Alveolar organoids derived from three lines of lung organoids were inoculated with SARS-CoV-2. At the indicated hours post-infection, culture media were harvested from AlvOs and applied to viral load detection and viral titration by TCID50 assay. Data represent mean and s.d. of one organoid line, n=3. (FIG. 2B) Representative confocal images of SARS-CoV-2 infected SFTPB+ AT2 cells in an AlvO. Nuclei and actin filaments were counterstained with DAPI (blue) and Phalloidin-647 (white), respectively. Scale bar, 20 μm. (FIG. 2C). AlvOs were subjected to immunofluorescence staining to label HTII-280+ AT2 cells and ACE2. Nuclei and actin filaments were counterstained with DAPI (blue) and Phalloidin-647 (white), respectively. Scale bar, 20 μm.

FIG. 3 is schematic summary of respiratory organoids.

### DETAILED DESCRIPTION OF THE INVENTION

### I. Definitions

A “base media,” as used herein, refers to a basal salt nutrient or an aqueous solution of salts and other elements that provide cells with water and certain bulk inorganic ions essential for normal cell metabolism and maintains intra-cellular and/or extra-cellular osmotic balance.

The term “Induced pluripotent stem cell” (iPSC), as used herein, is a type of pluripotent stem cell artificially derived from a non-pluripotent cell.

“Media” or “culture media” as used herein refers to an aqueous based solution that is provided for the growth, viability, or storage of cells used in carrying out the present invention. A media or culture media may be natural or artificial. A media or culture media may include a base media and may be supplemented with nutrients (e.g., salts, amino acids, vitamins, trace elements, antioxidants) to promote the desired cellular activity, such as cell viability, growth, proliferation, and/or differentiation of the cells cultured in the media.

“Organoid” as used herein refers to an artificial, in vitro construct derived from adult stem cells created to mimic or resemble the functionality and/or histological structure of an organ or portion thereof.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

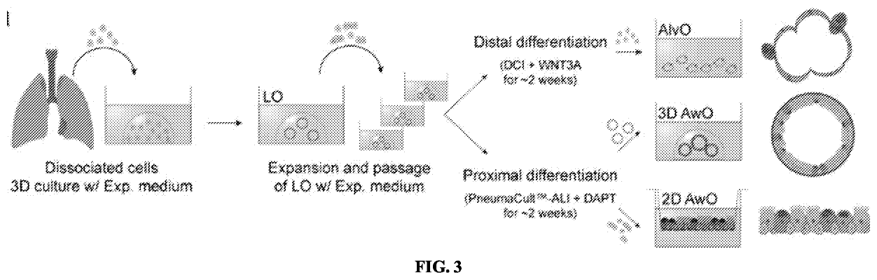

Human alveolar organoids have been derived from pluripotent stem cells (Kim, et al. Cell Death Discov. 7, 48 (2021). However, it is well known that pluripotent stem cells derived organoids are fetal-like in nature, which are unable to reach the maturation status of our alveolar organoids derived from adult stem cells in human lung tissues. Salahudeen, et al. Nature 588, 670-675 (2020) disclose methods for generating AT2 organoids. However, the organoids made according to the method disclosed in Salahudeen possess AT2 cells only, without AT1 cells, one of the major cell types in human alveolar epithelium. Hence, these AT2 organoids obtained following the protocol in Salahudeen are less physiologically-relevant than the alveolar organoids disclosed herein, which encompass both AT1 and AT2 cells. Secondly, in the Salahudeen culture system, a cell purification procedure is required to purify AT2 cells, followed by expansion of purified cells, to generate AT2 organoids. In contrast, none of these tedious purification or enrichment procedures is involved to generate the disclosed alveolar organoids. The disclosed alveolar organoids are directly and seamlessly derived from long-term expandable lung organoids (LO). The long-term expandable lung organoids serve as seeds and proliferate almost infinitely. Distal differentiation is induced in lung organoids (LO) to generate alveolar organoids (AlvO), and proximal differentiation is induced to produce airway organoids (AwO) (FIG. 3)). Briefly, long-term expandable lung organoids are digested into single cells and then switched to distal differentiation culture. after suspension culture in a non-adherent plate with distal differentiation medium for two weeks, the physiologically-active alveolar organoids are ready for various applications.

### II. Compositions

3D alveolar organoids and compositions for making them are provided. An organoid is a cellular cluster derived from stem cells or primary tissues and exhibits endogenous organ architecture (Cantrell and Kuo, Genome Medicine 7: 32-34 (2015)). Organoids differ from naturally occurring in vivo tissues and from ex vivo tissue explants in that they are derived from expansion of epithelial tissue cells only.

The 3D alveolar organoids include a population of AT1 and AT2 cells, express markers characteristic of the alveolar epithelium, appear morphologically similar to lung alveolar cells, and demonstrate functions characteristic of the mature alveolar epithelium.

### A. Alveolar Organoids

3D alveolar organoids are obtained (by cell culture) from cells dissociated from cells LO, which are subjected to a distal differentiation protocol in DD cell culture medium. In particularly preferred embodiments the disclosed alveolar organoids do not recombinantly express of Oct3/4, Sox2, Klf4, c-Myc, L-MYC, LIN28, shRNA for TP53 or combinations thereof, i.e., the alveolar organoids do not include cells genetically engineered to express Oct3/4, Sox2, Klf4, c-Myc, L-MYC, LIN28, shRNA for TP53 or combinations thereof.

The disclosed alveolar organoids are characterized in that compared to the lung organoids (LO) under expansion culture, the organoids of the same line applied to distal differentiation protocol disclosed herein show a significant upregulation (at least 10 fold higher) of canonical AT1 cell markers AQP5, HOPX and AT2 cell markers SFTPA, SFTPB, SFTPC, SFTPD. Compared to less than 10% AT1 cells and less than 30% AT2 in LO, AQP5+ AT1 cells and SFTPC+AT2 cells increased up to about 60% and about 40% respectively in the differentiated alveolar organoids. Alveolar organoids in suspension culture exhibit an apical-out polarity, as demonstrated by the expression of AQP5 and HTII-280, which are membrane proteins that are intrinsically expressed on the apical surface of AT1 and AT2 cells, respectively. Under transmission electron microscopy the presence of AT2 cells in the disclosed alveolar organoids can be seen with abundant cytoplasmic lamellar bodies and microvilli, as well as the presence of AT1 cells exhibiting a thin and flat morphology, characteristic of AT1 cells in vivo. AT2 cells in the disclosed alveolar organoids are further characterized in their ability to uptake surfactant and phospholipids.

3D alveolar organoids as disclosed herein are distinct from the differentiated airway organoids disclosed in WO 2019/228516 at least with respect to the cell population in the organoids, in that alveolar organoids do not include ciliated cells, which can be identified using ciliated cell markers (FOXJ1 and SNTN).

### III. Methods of Making the Composition/Device/Formulation

The disclosed methods include digesting long-term expandable lung organoids (LO) into single cells, culturing the digested cells in distal differentiation (DD) cell culture media supplemented with DCI, followed by suspension culture in a non-adherent plate with distal differentiation medium for an effective amount of time, following which, physiologically-active alveolar organoids are obtained.

The cells are cultured in DD media for a period of time ranging from preferably from 12-21 days, such as 12, 13, 14, 15, 16, 17, 18, 19, 20, or 21 days, preferably for at least 14 days.

### A. Sources of Cells for Culture

Lung organoids are obtained preferably, from a lung tissue sample from a subject. Briefly, the methods include obtaining a lung tissue sample from a subject, obtaining dissociated cells from the lung sample, culturing the dissociated cells in 3D culture in an expansion medium for an effective amount of time to obtain lung organoids, which are then long term expanded and passaged with expansion medium (FIG. 3).

The LO can be cultured from a tissue sample preferably a lung tissue sample obtained from a mammal, such as any mammal (e.g., bovine, ovine, porcine, canine, feline, equine, primate), preferably a human. In a preferred embodiment, the lung tissue is not obtained from an embryonic human lung and is preferably obtained from non-embryonic lungs for example, lung tissue from pediatric or adult subjects.

Single cells are obtained from a lung tissue sample using a combination of steps that result in single cells. The tissue sample size can range in size from 0.1 cm to 10 cm, for example, between 0.5 and 5 cm, in some preferred embodiments between 0.5 and 1.0 cm in size. Cells may be isolated by disaggregating an appropriate organ or tissue that is to serve as the cell source using techniques known to those skilled in the art. For example, the tissue or organ can be disaggregated mechanically and treated with digestive enzymes and/or chelating agents to release the cells, to form a suspension of individual cells. Enzymatic dissociation can be accomplished by mincing the tissue and treating the minced tissue with one or more enzymes such as trypsin, chymotrypsin, collagenase, elastase, and/or hyaluronidase, DNase, proteinase, dispase etc.

Isolated cells are further cultured as discussed herein. A preferred cell culture medium is a defined synthetic medium, buffered at a pH of 7.4 (preferably between 7.2 and 7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

The disclosed method generally includes: (a) obtaining a lung tissue sample from a subject, (b) isolating cells from the mammalian tissue to provide isolated cells by subjecting the tissue sample into single cells; (c) culturing the cells in an expansion culture medium for at least two to six weeks, preferably between three and four weeks to generate 3D lung organoids. The established 3D lung organoids can be maintained in expansion medium and passaged every two to three weeks. In step (d), lung organoids are subsequently cultured in differentiation medium, preferably a distal differentiation medium (DD), for a time sufficient for differentiation into alveolar organoids (AlvO). Steps (c) and (d) are preferably three dimensional (3D) cell culture, as opposed to 2D cell culture. While the 2D culture usually grows cells into a monolayer on glass or, more commonly, tissue culture polystyrene plastic flasks, 3D cell cultures grow cells into 3D aggregates/spheroids using a scaffold/matrix. Commonly used scaffold/matrix materials include biologically derived scaffold systems and synthetic-based materials.

In some preferred embodiment, the method is performed with a commercially using extracellular matrix. In some preferred embodiment, the method is performed with a commercially available extracellular matrix such as MATRIGEL™. (growth Factor Reduced Basement Membrane Matrix). Other extracellular matrices (ECM) are known in the art for culturing cells. A preferred ECM for use in a method of the invention includes at least two distinct glycoproteins, such as two different types of collagen or a collagen and laminin. In some preferred embodiment, the method is performed with a commercially available extracellular matrix such as MATRIGEL™. (growth Factor Reduced Basement Membrane Matrix), which comprises laminin, entactin, and collagen IV. In general, an extracellular matrix comprises laminin, entactin, and collagen. In a preferred embodiment, the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. Other commercially available products include Cultrex™ basement membrane extract (BME; Trevigen), and hyaluronic acid are commonly used biologically derived matrixes. Polyethylene glycol (PEG), polyvinyl alcohol (PVA), polylactide-co-glycolide (PLG), and polycaprolactone (PLA) are common materials used to form synthetic scaffolds. Scaffold-free 3D cell spheroids can be generated in suspensions by the forced floating method, the hanging drop method, or agitation-based approaches. Edmondson, et al., Assay Drug. Dev. Technol., 12(4):207-218 (2014). For example, the isolated cells are embedding in 60% MATRIGEL™ and seeded in a suspension culture plate prior to culture in the expansion medium.

In still another preferred embodiment, the expansion culture medium step does not include cells expressing Oct4 and/or are not genetically engineered to express one or more markers of pluripotency i.e., the cells iPSC, for example, adult cells induced to pluripotency by expression of Oct3/4, Sox2, Klf4, c-Myc, L-MYC, LIN28, shRNA for TP53 or combinations thereof, or embryonic stem cells, for example, H9 hESCs (Thomson et al., Science 282:1145-1147 (1998)), 201B7 (Takahashi et al., Cell, 131(5):861-72 (2007)), 585A1 or 604A1 hiPSCs (Okita et al., Stem Cells, 31(3):458-66 (2013)).

### B. Culture Media

The disclosed methods use basal cell culture media, supplemented with defined factors as disclosed herein to form expansion and differentiation media.

### i. Base Media

The cells are cultured in supplemented basal cell culture media. In some embodiments, a base media may include at least one carbohydrate as an energy source and/or a buffering system to maintain the medium within the physiological pH range. Examples of commercially available base media may include, but are not limited to, phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5 A Medium, Medium 199, William's Medium E, insect media such as Grace's medium, Ham's Nutrient mixture F-10 (Ham's F-10), Ham's F-12, a-Minimal Essential Medium (aMEM), Glasgow's Minimal Essential Medium (G-MEM) and Iscove's Modified Dulbecco's Medium. A preferred basal cell culture medium is selected from DMEM/F12 and RPMI 1640. In a further preferred embodiment, Advanced DMEM/F12 or Advanced RPMI is used, which is optimized for serum free culture and already includes insulin. In this case, the Advanced DMEM/F 12 or Advanced RPMI medium is preferably supplemented with glutamine and Penicillin/streptomycin.

In preferred embodiments, the basal medium comprises Gastrin. In some embodiments, the basal medium also comprises NAc and/or B27.

### ii. Expansion Media

In some embodiments an expansion medium as described in WO2016/083613 (referred to therein as AO medium) can be used. In a particularly preferred embodiment, an expansion culture medium (Table 1) is used, which is supplemented base media suitable to maintain lung organoids in culture.

The expansion culture medium is base medium supplemented with agents such as R-spondin (a Wnt agonist), a BMP inhibitor, a TGF-beta inhibitor, a fibroblast growth factor (FGF) and Nicotinamide. In some embodiments, the supplemented basal culture medium used to culture cells dissociated from a tissue sample does not include a GSK3 inhibitor, for example CHIR99021 (6-[[2-[[4-(2,4-Dichlorophenyl)-5-(5-methyl-1H-imidazol-2-yl)-2-pyrimidinyl]amino]ethyl]amino]-3-pyridinec arbonitrile). Known GSK-inhibitors comprise small-interfering RNAs, 6-Bromoindirubin-30-acetoxime.

TABLE 1
Composition of an expansion medium.

| | | | Working | |
|---|---|---|---|---|
| Reagents | Company | Catalog No. | concentration | |
| Advanced DMEM/F12 | Invitrogen | 12634010 | n/a | |
| HEPES | Invitrogen | 15630-056 | 1% | |
| GlutaMAX | Invitrogen | 35050061 | 1% | |
| Penicillin-Streptomycin | Invitrogen | 15140-122 | 1% | |
| Rspondin1* | n/a | n/a | 10% | |
| (conditioned medium) | | | | |
| Noggin* | n/a | n/a | 10% | |
| (conditioned medium) | | | | |
| B27 supplement | Invitrogen | 17504-044 | 2% | |
| N-acetylcysteine | Sigma | A9165 | 1.25 | mM |
| Nicotinamide | Sigma | N0636 | 10 | mM |
| Y-27632 | Tocris | 1254 | 5 | μM |
| A8301 | Tocris | 2939 | 500 | nM |
| SB202190 | Sigma | S7067 | 1 | μM |
| FGF-7 | Peprotech | 100-19 | 5 | ng/ml |
| FGF-10 | Peprotech | 100-26 | 20 | ng/ml |
| Primocin | InvivoGen | ant-pm-1 | 100 | μg/ml |
| Heregulin beta-1 | Peprotech | 100-03 | 5 | nM |

*Conditioned media were produced from stable cell lines for production of R-spondin1 and Noggin.

The expansion medium incudes a BMP inhibitor. BMP inhibitor is defined as an agent that binds to a BMP molecule to form a complex wherein the BMP activity is neutralized, for example by preventing or inhibiting the binding of the BMP molecule to a BMP receptor. Alternatively, the inhibitor is an agent that acts as an antagonist or reverse agonist. BMP-binding proteins that can be used in the disclosed methods include, but are not limited to Noggin (Peprotech), Chordin and chordin-like proteins (R&D systems) comprising chordin domains, follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems). Most preferred BMP inhibitor is Noggin. Noggin is preferably added to the basal culture medium at a concentration of at least about 10%.

The expansion medium incudes a WNT agonist. Wnt agonists include the R-spondin family of secreted proteins, which is include of 4 members (R-spondin 1 (NU206, Nuvelo, San Carlos, Calif.), R-spondin 2 ((R&D systems), R-spondin 3, and R-spondin-4); and Norrin. In a preferred embodiment, a Wnt agonist is selected from the group consisting of: R-spondin, Wnt-3a and Wnt-6. Preferred concentrations for the Wnt agonist are about 10% for R-spondin and approximately 100 ng/ml or 100 ng/ml for WNt-3a. In some preferred embodiments, the WNT agonist is not a GSK inhibitor.

SB202190 (4-(4-Fluorophenyl)-2-(4-hydroxyphenyl)-5-(4-pyridyl)-1H-imidazole) is a highly selective, potent and cell permeable inhibitor of p38 MAP kinase. SB 202190 binds within the ATP pocket of the active kinase (Kd=38 nM, as measured in recombinant human p38), and selectively inhibits the p38a and β isoforms. Other useful p38 MAPK inhibitors include, but are not limited to SB203580 (4-[5-(4-Fluorophenyl)-2-[4-(methylsulfonyl)phenyl]-1H-imidazol-4-yl]pyridine); SB 203580 hydrochloride (4-[5-(4-Fluorophenyl)-2-[4-(methylsulphonyl)phenyl]-1H-imidazol-4-yl]pyridine hydrochloride); SB202190 (4-[4-(4-Fluorophenyl)-5-(4-pyridinyl)-1H-imidazol-2-yl]phenol); DBM 1285 dihydrochloride (N-Cyclopropyl-4-[4-(4-fluorophenyl)-2-(4-piperidinyl)-5-thiazolyl]-2-pyrimidinamine dihydrochloride); SB 239063 (trans-4-[4-(4-Fluorophenyl)-5-(2-methoxy-4-pyrimidinyl)-1H-imidazol-1-yl]cyclohexanol); SKF 86002 dihydrochloride (6-(4-Fluorphenyl)-2,3-dihydro-5-(4-pyridinyl)imidazo[2,1-b]thiazole dihydrochloride).

A8301 (3-(6-Methyl-2-pyridinyl)-N-phenyl-4-(4-quinolinyl)-1H-pyrazole-1-carbothioamide) is potent inhibitor of TGF-0 type I receptor ALK5 kinase, type I activin/nodal receptor ALK4 and type I nodal receptor ALK7. A83-01 may be added to the culture medium at a concentration of between 10 nM and 10 μM, or between 20 nM and 5 μM, or between 50 nM and 1 μM. For example, A83-01 may be added to the culture medium at approximately 500 nM. Other useful TGF-0 type I receptor inhibitors include, but are not limited to SB431542 (4-[4-(1,3-benzodioxol-5-yl)-5-(2-pyridinyl)-1H-imidazol-2-yl]benzamide); LY 364947 (4-[3-(2-Pyridinyl)-1H-pyrazol-4-yl]-quinoline); R 268712 (4-[2-Fluoro-5-[3-(6-methyl-2-pyridinyl)-1H-pyrazol-4-yl]phenyl]-1H-pyrazole-1-ethanol); SB 525334 (6-[2-(1,1-Dimethylethyl)-5-(6-methyl-2-pyridinyl)-1H-imidazol-4-yl]quinoxaline); and SB 505124 (2-[4-(1,3-Benzodioxol-5-yl)-2-(1,1-dimethylethyl)-1H-imidazol-5-yl]-6-methyl-pyridine)

Y-27632 (trans-4-[(1R)-1-Aminoethyl]-N-4-pyridinylcyclohexanecarboxamide dihydrochloride) is a selective p160ROCK inhibitor. Other useful Rho inhibitors include isoquinolin and (S)-(+)-2-methyl-1-[(4-methyl-5-isoquinolinyl)sulfonyl]-hexahydro-1H-1,4-diazepine dihydrochloride (H-1152; Tocris Bioscience).

In particularly preferred embodiments, the expansion culture media used in the disclosed methods includes an ErbB3/4 ligand (e.g., human neuregulin β-1). The ErbB receptor tyrosine kinase family consists of four cell surface 5 receptors, ErbB1/EGFR HER1, ii) ErbB2/HER2, iii) ErbB3/HER3, and iv) ErbB4/HER4. ErbB3/4 ligands include members of the neuregulin/heregulin family. The neuregulin/heregulin family is referred to herein as the neuregulin family. The neuregulin family is a family of structurally related polypeptide growth factors that are gene products of alternatively spliced genes NRG1, NRG2, NRG3 and NRG4. In more preferred embodiments, the excluded one or more ErbB3/4 ligands of the culture medium are polypeptides that are gene products of one or more of NRG1, NRG2, NRG3 and NRG4 {i.e. a neuregulin polypeptide).

### iii. Distal Differentiation (DD) Medium

Distal differentiation medium is made from cell culture medium such as DMEM, Advanced DMEM/F-12 supplemented with 1% HEPES, 1% GlutaMAX, 1% Penicillin/Streptomycin. DD medium is preferably supplemented with maturation additives DCI (dexamethasone, 8-bromo-cyclic AMP, 3-isobutyl-1-methylxanthine), and WNT3A. The cell culture medium can be any basal medium as disclosed herein, however, Advanced DMEM/F-12 supplemented with 1% HEPES, 1% GlutaMAX, 1% Penicillin/Streptomycin is preferred.

Thus the cell culture medium is supplemented with effective amounts of a corticosteroid such as dexamethasone a cyclic AMP (cAMP) agonist such as 8-bromo-cyclic AMP, a cAMP/cGMP phosphodiesterase inhibitor such as 3-isobutyl-1-methylxanthine 3-isobutyl-1-methylxanthine?]]] and a Wnt agonist.

Dexamethasone is used at a concentration between about 25 and 150 nM, preferably between 40 to about 100 nM, and more preferably, between about 45 to 60 nM, for example, about 50 nM or about 55 nM.

cAMP agonists are known, and include, but are not limited to forskolin, prostaglandin E2 (PGE2), rolipram, genistein and cAMP analogs such as DBcAMP, and 8-bromocyclic AMP (used at a concentration between about 50 and about 500 μM, preferably between about 75 to 250 μM, more preferably, between 90 and 150 μM, for example about 95, about 100, about 105, about 110 μM etc.).

3-isobutyl-1-methylxanthine/IBMX is a non-selective, non-specific inhibitor of cAMP and cGMP phosphodiesterases. IBMX is used at a concentration between about 50 and about 500 μM, preferably between about 75 to 250 μM, more preferably, between 90 and 150 μM, for example about 95, about 100, about 105, about 110 μM etc.

Wnt agonists include the R-spondin family of secreted proteins, which is include of 4 members (R-spondin 1 (NU206, Nuvelo, San Carlos, Calif.), R-spondin 2 ((R&D systems), R-spondin 3, and R-spondin-4); and Norrin. In a preferred embodiment, a Wnt agonist is selected from the group consisting of: R-spondin, Wnt-3a and Wnt-6, with Wnt3 being particularly preferred. Preferred concentrations for the Wnt agonist are about 25-60% v/v; preferably 30-55% for example, 30, 40, 45, 50, 55% v/v for WNt-3a. In some preferred embodiments, the WNT agonist is not a GSK inhibitor.

The DD medium preferably does not include a notch inhibitor, such as DAPT or dibenzazepine (DBZ) or benzodiazepine (BZ) or LY-411575, an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases, which are used in methods for proximal differentiation of LO as disclosed in WO 2019/228516.

A particularly preferred Distal differentiation (DD) medium includes Advanced DMEM/F-12 supplemented with 1% HEPES, 1% GlutaMAX, 1% Penicillin/Streptomycin, 50 nM dexamethasone, 100 μM 8-bromo-cAMP, 100 μM IBMX, 2% B-27, 50% Wnt3A conditioned medium.

### IV. Methods of Using

The methods discussed above provide a novel, robust and easy-to-operate culture system, whereby alveolar organoids can be made and readily-maintained in vitro. These alveolar organoids can be utilized alone or in combination with 2D and 2D AWO (FIG. 3), to study biology and pathology of human respiratory system, including, but not limited to, the study of influenza virus infections and COVID-19 respiratory diseases. The virus can be a Severe acute respiratory syndrome-related coronavirus, a Bat Hp-beta coronavirus Zhejiang2013, a Rousettus bat coronavirus GCCDC1, a Rousettus bat coronavirus HKU9, Eidolon bat coronavirus C704, a Pipistrellus bat coronavirus HKU5, a Tylonycteris bar coronavirus HKU4, a Middle East respiratory syndrome-related coronavirus, a Hedgehog coronavirus, a murine coronavirus, a Human coronavirus HKU1, a China Rattus coronavirus HKU24, a Beta coronavirus 1, a Myodes coronavirus 2JL14, a Human coronavirus NL63, a Human coronavirus 229E, or a Human coronavirus OC43.

In some embodiments, the virus is a Severe acute respiratory syndrome-related coronavirus, such as SARS-CoV-2, SARS-CoV, SARSr-CoV RaTG13, SARS-CoV PC4-227, or SARSr-CoV BtKY72.

In some embodiments, the virus is a severe acute respiratory syndrome coronavirus 2 (SARS-CoV-2) having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity SE.

Organoids derived from adult stem and progenitor cells reliably retain their in vivo regenerative activity in vitro, and thus provide detailed snapshots of tissue repair after injury. Lung organoids allow researchers to study processes governing homeostatic regulation of lung tissue and screen factors that impact lineage-specification of stem cells.

Accordingly, the disclosed alveolar organoids may be used as an alternative to live animal testing for compound or for treatment of (including resistance to treatment of) lung infection or disease (e.g., chronic obstructive pulmonary disease (COPD).

In some preferred embodiment, the disclosed alveolar organoid can be used for influenza virus testing (infectivity). In a particularly preferred embodiment, the disclosed alveolar organoids can discriminate human infective influenza viruses from poorly infective viruses. Thus, the distal differentiated lung organoids can be utilized to predict the infectivity of influenza viruses and significantly extend the current armamentaria of influenza research toolbox.

Pre-clinical models of human disease are essential for the basic understanding of disease pathology and its translational application into efficient treatment for patients. Patient-derived organoid cultures from biopsies and/or surgical resections can be used for personalized medicine. Two examples are lung cancer and cystic fibrosis. Additionally, tissue samples can be obtained from a subject cultured as disclosed herein and used to determine the subject's responsiveness to medication in order to select the better treatment for that subject. Dekkers et al. Science Translational Medicine, 8(344):344ra84 (2016) showed that the efficacy of cystic fibrosis transmembrane conductance regulator (CFTR)-modulating drugs can be individually assessed in a laboratory test using epithelial cells cultured as mini-guts from rectal biopsies from subjects with cystic fibrosis. The authors show that the drug responses observed in mini-guts or rectal organoids can be used to predict which patients may be potential responders to the drug. Similar preclinical tests using the disclosed 3D organoids obtained from a subject may help to quickly identify responders to CFTR-modulating drug therapy even when patients carry very rare CFTR mutations.

Ex vivo expanded adult stem cell-derived organoids retain their organ identity and genome stability and can be differentiated to alveolar organoids as described herein. Therefore, the alveolar organoids may also be used for replacing damaged tissues.

The disclosed compositions and methods can be further understood through the following numbered paragraphs.

1. A method of generating an alveolar organoid (AlvO) comprising culturing a lung organoid (LO) in a distal differentiation medium for a period sufficient to generate an AlvO comprising a cell population consisting of at least
40% type I alveolar epithelial (AT1) cells and type II alveolar epithelial (AT2) cells, wherein the AT1 cells are AQP5+ and the AT2 cells are SFTPC+;
wherein the distal differentiation medium is supplemented with an effective amount of (i) dexamethasone, (ii) a cyclic AMP (cAMP) agonist such as 8-bromo-cyclic AMP, (iii) a cAMP/cGMP phosphodiesterase inhibitor such as 3-isobutyl-1-methylxanthine 3-isobutyl-1-methylxanthine?]]] and a (iv) Wnt agonist.
   2. The method of paragraph 1, wherein the Wnt agonist is Wnt3.
   3. The method of paragraph 2, wherein the Wnt3 is at a concentration of about 25-60% v/v; preferably 30-55% for example, 30, 40, 45, 50, 55% v/v.
   4. The method of claim 3, wherein IBMX is used at a concentration between about 50 and about 500 μM, preferably between about 75 to 250 μM, more preferably, between 90 and 150 μM, for example about 95, about 100, about 105, about 110 μM etc.
   5. The method of any one of the preceding paragraph s, wherein (a) 8-bromocyclic AMP is at concentration between about 50 and about 500 PM, preferably between about 75 to 250 μM, more preferably, between 90 and 150 μM, for example about 95, about 100, about 105, about 110 μM; and/or (b) dexamethasone is used at a concentration between about 25 and 150 nM, preferably between 40 to about 100 nM, and more preferably, between about 45 to 60 nM, for example, about 50 nM or about 55 nM.
   6. The method of any one of the preceding paragraph s, wherein the method further comprises one or more of the following steps prior to culturing the LO in a DD medium:
a. obtaining a lung tissue sample from a subject;
b. obtaining dissociated cells from a lung tissue sample; and
c. culturing lung cells in an lung organoid (LO) formation phase to form a lung organoid (LO) for a period sufficient to generate a LO.
   7. The method of paragraph 6, wherein the LO formation phase comprises
culturing cells in an expansion medium comprising one or more components as set out in Table 1, optionally at the concentrations shown in Table 1.
   8. The method of paragraph 7, wherein the expansion medium comprises at least R-spondin, a BMP inhibitor, a TGF-beta inhibitor, FGF and heregulin beta-1.
   9. The method of any one of the preceding paragraph s, wherein the step of
culturing the lung cells and/or LO comprises culturing the cells in contact with an exogenous extracellular matrix.
   10. The method of any one of the preceding paragraphs, wherein the LO is a 3D organoid.
   11. The method of any one of the preceding paragraphs, wherein the AlvO is a 3D organoid.
   12. A method of generating a AlvO in accordance with any one of paragraphs 1 to 11 comprising the steps of:
a. culturing lung cells from a subject in LO formation phase in an expansion medium in contact with an extracellular matrix for a period sufficient to generate a 3D LO, for example for at least 2 four weeks and
b. changing the expansion medium to a distal differentiation medium supplemented with a notch inhibitor and culturing the 3D LO-organoid in the distal differentiation medium comprising a an effective amount of dexamethasone, a cyclic AMP (cAMP) agonist such as 8-bromo-cyclic AMP, a cAMP/cGMP phosphodiesterase inhibitor such as 3-isobutyl-1-methylxanthine and a Wnt agonist for a period sufficient to generate an AlvO, for example for at least 5 days, at least 10 days, at least 14 days or at least 16 days.
   13. The method of any one of the preceding paragraphs, wherein the culture medium is refreshed every other day.
   14. The method of any one of the preceding paragraphs, wherein the organoid or cells are human organoids or human cells.
   15. An ALvO obtained by a method of any one of paragraphs 1 to 14, wherein the AlvO comprises a cell population consisting of at least 40% type I alveolar epithelial (AT1) cells and type II alveolar epithelial (AT2) cells, wherein the AT1 cells are AQP5+ and the AT2 cells are SFTPC+.
   16. The AlvO of paragraph 15, wherein the AlvO has at least 2-fold or at least 3-fold upregulation in AT1 cell marker AQP5 or HOPX and/or AT2 cell markers selected from the group consisting of SFTPA, SFTPB, SFTPC, and SFTPD compared to the LO from which they are obtained.
   17. The AlvO according to any one of paragraphs 1-16, wherein the AlvO in suspension culture exhibit an apical-out polarity, as demonstrated by the expression of AQP5 and HTII-280.
   18. The AlvO according to any one of paragraphs 16-17, wherein gene expression is assessed using quantitative PCR of mRNA transcripts normalized with GAPDH.
   19. The AlvO of any one of paragraphs 15-18, wherein the AlvO further comprises a test virus.
   20. A method for contacting a virus in a AlvO, comprising:
a. generating an AlvO in accordance with any one of claims 1-19; and
b. infecting the AlvO with the test virus, optionally, wherein the text virus is an influenza virus or coronavirus.
   21. The method of paragraph 20, wherein the infecting step further comprising incubating for at least 30 minutes, at least 60 minutes, at least 90 minutes or at least 120 minutes.
   22. The method of paragraph 21, wherein the incubating step is performed at about 37° C.
   23. A method for predicting infectivity of a test virus to humans, comprising:
a. generating a human AlvO in accordance with any one of paragraphs 1-19;
b. contacting the human AlvO with the test virus;
c. testing the viral titre after a period sufficient to allow viral propagation
d. optionally comparing the viral titre to a control virus, herein, optionally, the text virus is influenza virus or coronavirus.
   24. The method of paragraph 23, wherein testing the viral titre involves detecting a change in viral titre.
   25. The method of paragraph 24, wherein an increase in viral titre is indicative of likely infectivity of the influenza virus to humans and/or wherein a greater increase over a shorter period is correlated with a higher degree of infectivity.
   26. The method of paragraph 25, wherein the increase in viral titre is at least 1 log 10 units, at least 2 log 10 units, or at least 3 log 10 units within 24 hours.
   27. The method of paragraph 26, wherein the control virus is a known poorly-infective-to-humans virus, optionally wherein the change in viral titre of the test virus is greater than the change in viral titre of the known poorly-infective-to-humans virus, for example wherein the viral titre is at least 10-fold, at least 50-fold, at least 100-fold, at least 1,000 fold or at least 10,000 fold greater than the viral titre of the known poorly-infective-to-humans virus.
   28. The method of paragraph 27, wherein the known poorly-infective influenza virus is selected from H7N2, H9N2 and H9N9 and the text virus is an influenza virus.
   29. The method of paragraph 28, wherein the control influenza virus is a known infective-to-humans influenza virus, optionally wherein the change viral titre of the test influenza virus is about the same or greater than the viral titre of the known infective-to-humans influenza virus, for example, at least 75%, at least 80%, at least 90%, at least 100%, at least 150%, at least 2-fold, at least 5-fold or at least 10-fold relative to the viral titre of the known infective-to-humans influenza virus.
   30. The method of paragraph 29, wherein the known infective influenza virus is H7N9.

The present invention will be further understood by reference to the following non-limiting examples.

### EXAMPLES

### Materials and Methods

Establishment, maintenance and differentiation of respiratory organoids After ethical approval by the Institutional Review Board, human lung organoids were established previously using the lung tissues from patients who underwent surgical resection for various diseases²⁰. Briefly, small pieces of normal lung tissues around 0.5-0.8 cm in size, which are adjacent to the diseased tissues, were obtained from patients who underwent surgical operation. The tissues were minced into small pieces and digested with 2 mg/ml collagenase (Sigma Aldrich) for 1-2 hours at 37° C., followed by shearing using glass Pasteur pipette and straining over a 100 μm filter. The resultant single cells were embedded in 60% Matrigel (Growth Factor Reduced Basement Membrane Matrix, Corning) and were seeded in 24-well suspension culture plate. After solidification, Matrigel droplets were maintained with expansion culture medium (Table 1) at 37° C. in a humidified incubator with 5% CO2. The organoids were passaged every 2-3 weeks. Bright field images of the organoids were acquired using Nikon Eclipse TS100 Inverted Routine Microscope.

### Expansion and Passage of LO

Human lung organoids are maintained in a previously defined expansion medium and passaged every 2-3 weeks²⁰.

### Distal Differentiation of LO Cells

Lung organoids were enzymatically digested using 10×TrypLE™ Select Enzyme (Invitrogen) for 1-5 min at 37° C., sheared using Pasteur pipette and strained over a 40 μm filter. Single cells were suspension cultured in the distal differentiation (DD) media supplemented with the alveolar maturation additives DCI (dexamethasone, 8-bromo-cyclic AMP, 3-isobutyl-1-methylxanthine), and WNT3A conditioned medium which were reported to drive alveolar differentiation, followed by incubation for 2 weeks. To direct distal differentiation into alveolar organoids, after treatment with 10× TrypLE™ Select (Invitrogen) for 5 minutes at 37° C., single cells dissociated from the lung organoids were then cultured in distal differentiation (DD) medium (Advanced DMEM/F-12 supplemented with 1% HEPES, 1% GlutaMAX, 1% Penicillin/Streptomycin, 50 nM dexamethasone, 100 uM 8-bromo-cAMP, 100 uM IBMX, 2% B-27, 50% Wnt3A conditioned medium), and 100 ug/ml Primocin in Nunclon Sphera super-low attachment plate (Thermo) at 37° C. in a humidified incubator with 5% C02. The medium was replenished every other day for 12-14 days. The undifferentiated lung organoids, and alveolar organoids were harvested and applied to RNA extraction using MiniBEST Universal RNA Extraction kit (Takara), followed by reverse transcription using Transcriptor First Strand cDNA Synthesis Kit (Roche) and oligo(dT) primer. The resultant cDNAs were used to measure mRNA expression levels of cellular genes using the LightCycler 480 SYBR Green I Master Mix (Roche).

### Inoculation of Alveolar Organoids with SARS-CoV-2 and Replication of SARS-CoV-2 in Alveolar Organoids

The differentiated human alveolar organoids were incubated with the SARS-CoV-2 for 2 hours at 37° C., followed by incubation in the basal medium. To assess replication kinetics, at the indicated hours after inoculation, cell-free culture media was harvested, followed by RNA extraction using the MiniBEST Viral RNA/DNA Extraction Kit (Takara) and detection of viral loads (viral gene copy number of RdRp) by one-step RT-qPCR assay, and viral titration by TCID₅₀ assay.

### Uptake of Surfactant and Phospholipids by AT2 Cells

The functionality of alveolar organoids is demonstrated by evaluating the capacity of AT2 cells to uptake surfactant and phospholipids, which is an important biological function of native AT2 cells³.

LysoTracker, a fluorescent dye that preferentially binds acidic organelles, can label the lamellar bodies and is commonly used for live cell imaging and cell sorting of AT2 cells 3, 4. The alveolar organoids were incubated in DD medium supplemented with a green fluorescent-labeled phosphocholine, β-BODIPY™ FL C12-HPC ((2-(4,4-Difluoro-5,7-Dimethyl-4-Bora-3a,4a-Diaza-s-Indacene-3-Dodecanoyl)-1-Hexadecanoyl-sn-Glycero-3-Phosphocholine)). After a further incubation with LysoTracker™ red (deep red-fluorescent dye for labeling and tracking acidic organelles in live cells), the organoids uptaking the fluorescent-labeled phosphocholine were monitored. Live cell confocal imaging clearly revealed the co-localization of the labeled phosphocholine with LysoTracker labeled AT2 cells.

### Immunofluorescence Staining and Flow Cytometry

After fixation with 4% PFA, permeabilization with 0.5% Triton X-100 and blocking with protein block (Dako), organoids were applied to immunofluorescence. Cellular compositions of the organoids were characterized by specific antibodies for AT1 cell (AQP5), AT2 cell (SFTPC, SFTPB, HTII-280) followed by secondary antibodies. Nuclei and actin filaments were counterstained with DAPI (Thermo Fisher Scientific) and Phalloidin-647 (Sigma-Aldrich), respectively. The organoids were then whole mounted on a glass slide with ProLong™ Glass Antifade Mountant (Invitrogen). The confocal images were acquired using a Carl Zeiss LSM 800 confocal microscope. For flow cytometry analysis, Organoids were dissociated into single cells with 10 mM EDTA (Invitrogen) for 30 minutes at 37° C. and then fixed with 4% PFA for 15 minutes at room temperature. A BD FACSCantoII Analyzer was used for analysis.

### Live-Cell Staining of Lamellar Bodies in AlvO Organoids

Differentiated human alveolar organoids were incubated with 1 uM β-BODIPY™ FL C12-HPC (D3792, Invitrogen) in DD medium for 24 hours at 37° C. After washing with basal medium, the organoids were incubated with 100 nM LysoTracker™ Red DND-99 (L7528, Invitrogen) in DD medium for 30 minutes at room temperature. The organoids were then counterstained with Hoechst 33342 (62249, Thermo) and CellMask™ Deep Red plasma membrane stain (C10046, Invitrogen) and applied to confocal imaging using a Carl Zeiss LSM 800 confocal microscope.

### Statistical Analysis

Student's t test was used for data analysis. P<0.05 was considered to be statistically significant.

### Results

### Distal Differentiation of the Lung Organoids

Briefly, after enzymatic digestion of lung organoid, single cells were suspension cultured in the distal differentiation (DD) media supplemented with the alveolar maturation additives DCI (dexamethasone, 8-bromo-cyclic AMP, 3-isobutyl-1-methylxanthine), and WNT3A conditioned medium which were reported to drive alveolar differentiation. After incubation for 2 weeks, single cells from the original lung organoids (which normally have a central lumen) grew into cellular clusters composed of cuboidal and thin cells, with a morphology reminiscent of alveoli.

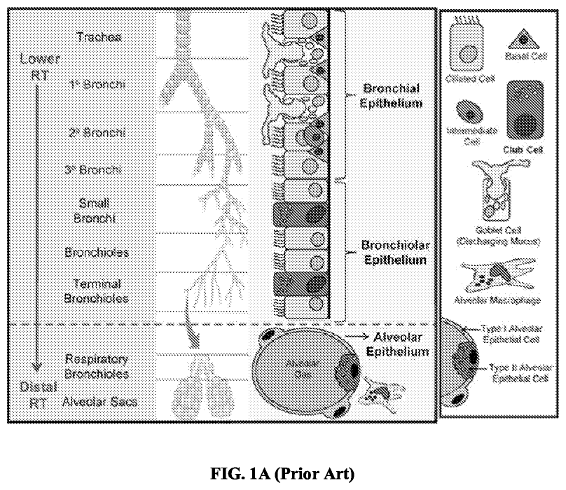

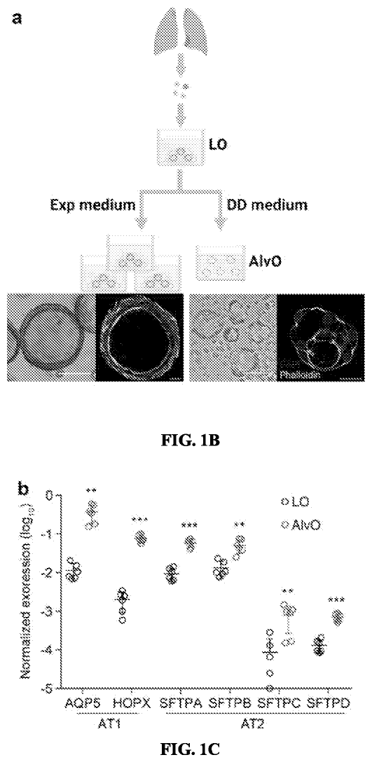

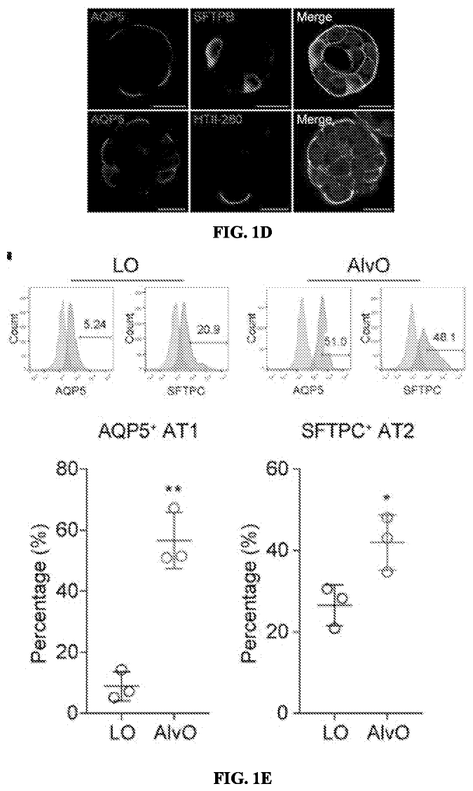

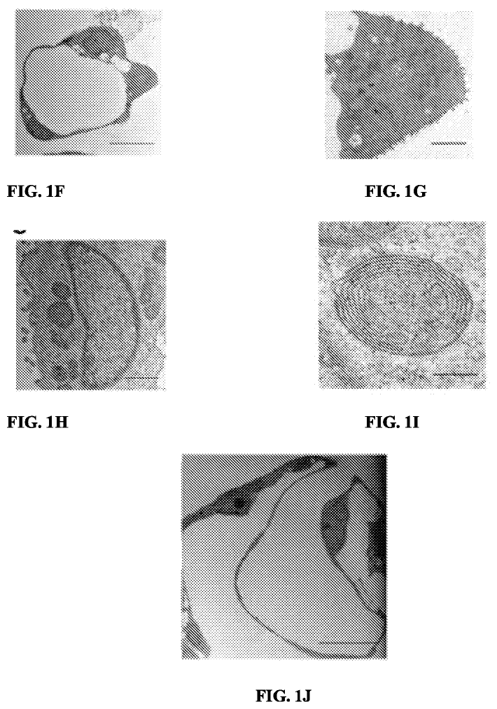

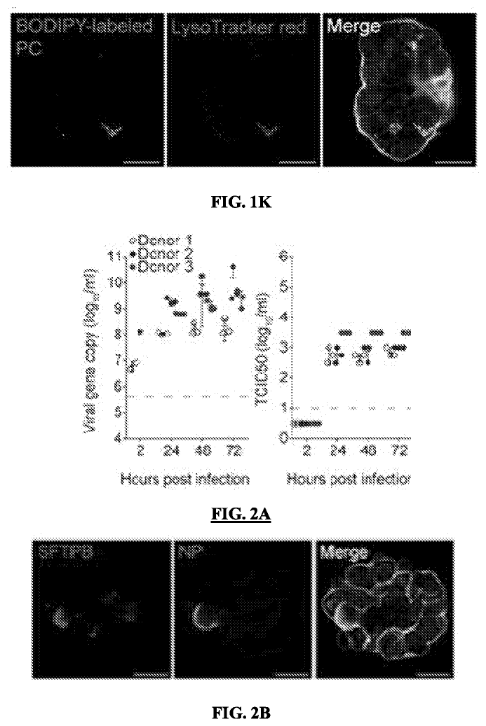

To direct lung organoids into alveolar organoids enriched with AT1 and AT2 cells, an array of growth factors essential for alveolar differentiation was and eventually settled on a distal differentiation protocol. Briefly, single cells were disassociated from lung organoids and suspension-cultured in the distal differentiation (DD) medium, in which Wnt3a conditioned medium and alveolar maturation additives, including dexamethasone, 8-bromo-cyclic AMP, 3-isobutyl-1-methylxanthine25, 26 were incorporated. After 2-week incubation, single cells from the lung organoids grew into cystic clusters composed of cuboidal and thin cells (FIG. 1B), a morphology reminiscent of alveoli. In contrast, these single cells proliferated when maintained in the expansion (Exp) medium, and formed typical lung organoids, which assume a morphology of thick walls and a central lumen (FIG. 1B).

Compared to the lung organoids (LO) of the same line maintained under expansion culture, the organoids subject to distal differentiation protocol exhibited a significant upregulation of canonical AT1 cell markers AQP5 (aquaporin 5), Hop Homebox (HOPX) and AT2 cell markers Surfactant, Pulmonary-Associated Protein (SFTP)A, SFTPB, SFTPC, SFTPD (FIG. 1C), which are termed alveolar organoids (AlvO). Abundant AT1 and AT2 cells in the alveolar organoids were clearly discernible in immunofluorescence staining (FIG. 1D). Consistent with a recent report in the intestinal organoids27, the alveolar organoids in suspension culture exhibited an apical-out polarity, which is demonstrated by the exterior localization of AQP5 and HTII-280, the membrane proteins intrinsically expressed on the apical membrane of AT1 and ATII cells respectively.

Flow cytometry analysis revealed that AQP5+ AT1 cell and SFTPC+AT2 became significantly enriched compared to those in the lung organoids and each increased to around 50% (compared to less than 10% AT1 cells and less than 30% AT2 in LO) and became significantly enriched than those in the lung organoids (FIG. 1E). Transmission electron microscopy showed organoids composed of AT1 and AT2 cells (FIG. 1F). AT2 cells are featured with microvilli (FIG. 1G) and cytoplasmic lamellar bodies (FIG. 1H, 1I), a secretory vesicle containing surfactant proteins. AT1 cells in the organoids exhibited a thin and flat morphology (FIG. 1J), a characteristic of AT1 cells in vivo. FIG. 1K shows the result of AlvOs subjected to immunofluorescence staining to label HTII-280+ AT2 cells and ACE2 expressing cells. Nuclei and actin filaments were counterstained with DAPI (blue) and Phalloidin-647 (white), respectively. Scale bar, 20 μm.

### Functionality of Alveolar Organoids

Subsequent studies assessed the functionality of alveolar organoids by evaluating the capacity of AT2 cells to uptake surfactant and phospholipids, which is an important biological function of native AT2 cells 3. LysoTracker, a fluorescent dye that preferentially binds acidic organelles, can label the lamellar bodies and is commonly used for live cell imaging and cell sorting of AT2 cells^{3, 4}. The alveolar organoids were incubated in DD medium supplemented with a green fluorescent-labeled phosphocholine, β-BODIPY FL C12-HPC or mock-treated with the DD medium only. After further incubation with LysoTracker red, the organoids were monitored by live-cell confocal imaging. Live cell confocal imaging clearly revealed the co-localization of the labeled phosphocholine with LysoTracker labeled AT2 cells. Co-localization of the green-florescent phosphocholine with LysoTracker labeled AT2 cells was observed (FIG. 1J), whereas the co-localization is absent in LysoTracker red labeled AT2 cells within the mock-treated organoids (results not shown). The results indicate that AT2 cells in the alveolar organoids are indeed functional and capable of uptake surfactant components.

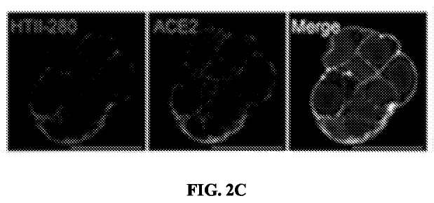

A productive SARS-CoV-2 infection in alveolar organoids from three different donors was observed. As shown in FIG. 2A, viral load and viral titer increased significantly after SARS-CoV-2 inoculation. In addition, immunofluorescence staining revealed the infection of SFTPB+ AT2 cells by SARS-CoV-2 (FIG. 2C). SARS-CoV-2 cellular receptor ACE2 is expressed on the membrane of AT2 cells. FIG. 2B shows representative confocal images of SARS-CoV-2 infected SFTPB+ AT2 cells in an AlvO. Nuclei and actin filaments were counterstained with DAPI (blue) and Phalloidin-647 (white), respectively.

### DISCUSSION

In summary, the previous studies have established a robust protocol to derive human lung organoids from primary lung tissues, that are stably expandable with expansion (Exp) medium in culture plates up to one year without any feeder cells. The long-term expandable lung organoids (LO) can be further applied to bidirectional differentiation, i.e. proximal differentiation to generate airway organoid (AwO) in 2-dimensional (2D) or 3-dimensional (3D) format or distal differentiation to derive alveolar organoids (AlvO) (FIG. 3). Collectively, a novel, robust and easy-to-operate culture system is provided, whereby the entire human respiratory epithelium is rebuilt and readily-maintained in vitro. These pulmonary organoids can be utilized to study biology and pathology of human respiratory system, including, but not limited to, the study of COVID-19 respiratory diseases.

The organoids derived from lung tissues (i.e., the long-term expandable lung organoids (LO)) were previously named “airway organoids’ since most organoids display thick walls with discernible beating cilia. Subsequent analysis revealed AT2 cells are present in the “airway organoids”, herein, LO, at a percentage of around 20-30%; these cells are therefore, more appropriately identified lung organoids. The presence of abundant AT2 cells in lung organoids is not surprising since R-spondin 1, an agonist of Wnt signaling as an Lgr5 ligand, was incorporated in the lung organoid expansion medium. Several groups demonstrated that that canonical Wnt signaling enables long-term AT2 cell expansion²⁹⁻³¹ In addition, high Wnt signaling drove AT2 cell proliferation and transdifferentiation into AT1 cells^{21,32-34}. Hence, distal differentiation in lung organoids was attempted resulting in successful generation of alveolar organoids enriched with AT1 and functional AT2 cells.

Here, a two-phase organoid culture system is disclosed that enables to expand the whole human respiratory epithelium stably in culture plates. Lung organoids are directly derived from somatic epithelial cells in primary human lung tissues and are long-term expanded in the expansion medium. Proximal or distal differentiation is induced in the expandable lung organoids to seamlessly generate airway organoids (AwO) or alveolar organoids (AlvO) respectively within two weeks (FIG. 3). During the whole procedure, including initial derivation, long-term expansion and bi-directional differentiation, neither tedious cell purification nor feeder and stromal cells are required. In addition, the distal differentiation protocol enables direct generation of alveolar organoids with both AT1 and AT2 cells, unlike other alveolar differentiation protocols, in which extra manipulation is needed to generate AT1 cells^{14, 18, 30} Collectively, novel, robust and easy-to-operate culture system has been established, thereby the entire human respiratory epithelium is rebuilt and readily maintained in vitro, in the combination of AwO and AlvO disclosed herein. These respiratory organoids can be utilized to study biology and pathology of the human respiratory system, including, but not limited to, the study of COVID-19 respiratory diseases.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

### REFERENCES

1. Zhou, P. et al. A pneumonia outbreak associated with a new coronavirus of probable bat origin. Nature 579, 270-273 (2020).
2. Wolfel, R. et al. Virological assessment of hospitalized patients with COVID-2019. Nature 581, 465-469 (2020).
3. Wang, D. et al. Clinical Characteristics of 138 Hospitalized Patients With 2019 Novel Coronavirus-Infected Pneumonia in Wuhan, China. JAMA (2020).
4. Carsana, L. et al. Pulmonary post-mortem findings in a series of COVID-19 cases from northern Italy: a two-centre descriptive study. Lancet Infect Dis 20, 1135-1140 (2020).
5. Rockx, B. et al. Comparative pathogenesis of COVID-19, MERS, and SARS in a nonhuman primate model. Science 368, 1012-1015 (2020).
6. Xu, Z. et al. Pathological findings of COVID-19 associated with acute respiratory distress syndrome. Lancet Respir Med 8, 420-422 (2020).
7. Schaefer, I. M. et al. In situ detection of SARS-CoV-2 in lungs and airways of patients with COVID-19. Mod Pathol 33, 2104-2114 (2020).
8. Hou, Y. J. et al. SARS-CoV-2 Reverse Genetics Reveals a Variable Infection Gradient in the Respiratory Tract. Cell 182, 429-446 e414 (2020).
9. Zhu, N. et al. Morphogenesis and cytopathic effect of SARS-CoV-2 infection in human airway epithelial cells. Nat Commun 11, 3910 (2020).
10. Zhou, B. et al. SARS-CoV-2 spike D614G change enhances replication and transmission. Nature 592, 122-127 (2021).
11. Butler, C. R. et al. Rapid Expansion of Human Epithelial Stem Cells Suitable for Airway Tissue Engineering. Am J Respir Crit Care Med 194, 156-168 (2016).
12. Clevers, H. Modeling Development and Disease with Organoids. Cell 165, 1586-1597 (2016).
13. Han, Y. et al. Identification of SARS-CoV-2 inhibitors using lung and colonic organoids. Nature (2020).
14. Katsura, H. et al. Human Lung Stem Cell-Based Alveolospheres Provide Insights into SARS-CoV-2-Mediated Interferon Responses and Pneumocyte Dysfunction. Cell Stem Cell (2020).
15. Youk, J. et al. Three-Dimensional Human Alveolar Stem Cell Culture Models Reveal Infection Response to SARS-CoV-2. Cell Stem Cell (2020).
16. Huang, J. et al. SARS-CoV-2 Infection of Pluripotent Stem Cell-Derived Human Lung Alveolar Type 2 Cells Elicits a Rapid Epithelial-Intrinsic Inflammatory Response. Cell Stem Cell (2020).
17. Salahudeen, A. A. et al. Progenitor identification and SARS-CoV-2 infection in human distal lung organoids. Nature (2020).
18. Salahudeen, A. A. et al. Progenitor identification and SARS-CoV-2 infection in human distal lung organoids. Nature 588, 670-675 (2020).
19. Lamers, M. M. et al. An organoid-derived bronchioalveolar model for SARS-CoV-2 infection of human alveolar type II-like cells. EMBO J 40, e105912 (2021).
20. Zhou, J. et al. Differentiated human airway organoids to assess infectivity of emerging influenza virus. Proc Natl Acad Sci USA 115, 6822-6827 (2018).
21. Sachs, N. et al. Long-term expanding human airway organoids for disease modeling. EMBO J 38 (2019).
22. Barkauskas, C. E. et al. Type 2 alveolar cells are stem cells in adult lung. J Clin Invest 123, 3025-3036 (2013).
23. Kumar, P. A. et al. Distal airway stem cells yield alveoli in vitro and during lung regeneration following H1N1 influenza infection. Cell 147, 525-538 (2011).
24. Guha, A., Deshpande, A., Jain, A., Sebastiani, P. & Cardoso, W. V. Uroplakin 3a(+) Cells Are a Distinctive Population of Epithelial Progenitors that Contribute to Airway Maintenance and Post-injury Repair. Cell Rep 19, 246-254 (2017).
25. Jacob, A. et al. Differentiation of Human Pluripotent Stem Cells into Functional Lung Alveolar Epithelial Cells. Cell Stem Cell 21, 472-488 e410 (2017).
26. Huang, S. X. et al. Efficient generation of lung and airway epithelial cells from human pluripotent stem cells. Nat Biotechnol 32, 84-91 (2014).
27. Co, J. Y. et al. Controlling Epithelial Polarity: A Human Enteroid Model for Host-Pathogen Interactions. Cell Rep 26, 2509-2520 e2504 (2019).
28. Yamamoto, Y. et al. Long-term expansion of alveolar stem cells derived from human iPS cells in organoids. Nat Methods 14, 1097-1106 (2017).
29. Nabhan, A. N., Brownfield, D. G., Harbury, P. B., Krasnow, M. A. & Desai, T. J. Single-cell Wnt signaling niches maintain stemness of alveolar type 2 cells. Science 359, 1118-1123 (2018).
30. Youk, J. et al. Three-Dimensional Human Alveolar Stem Cell Culture Models Reveal Infection Response to SARS-CoV-2. Cell Stem Cell 27, 905-919 e910 (2020).
31. Zacharias, W. J. et al. Regeneration of the lung alveolus by an evolutionarily conserved epithelial progenitor. Nature 555, 251-255 (2018).
32. Lee, J. H. et al. Anatomically and Functionally Distinct Lung Mesenchymal Populations Marked by Lgr5 and Lgr6. Cell 170, 1149-1163 e1112 (2017).
33. McCauley, K. B. et al. Efficient Derivation of Functional Human Airway Epithelium from Pluripotent Stem Cells via Temporal Regulation of Wnt Signaling. Cell Stem Cell 20, 844-857 e846 (2017).
34. Chung, M. I., Bujnis, M., Barkauskas, C. E., Kobayashi, Y. & Hogan, B. L. M. Niche-mediated BMP/SMAD signaling regulates lung alveolar stem cell proliferation and differentiation. Development 145 (2018).

1. A method of generating an alveolar organoid (AlvO) comprising culturing a lung organoid (LO) in a distal differentiation medium for a period sufficient to generate an AlvO comprising a cell population consisting of at least 40% type I alveolar epithelial (AT1) cells and type II alveolar epithelial (AT2) cells, wherein the AT1 cells are AQP5+ and the AT2 cells are SFTPC+;
wherein the distal differentiation medium is supplemented with an effective amount of (i) dexamethasone, (ii) a cyclic AMP (cAMP) agonist such as 8-bromo-cyclic AMP, (iii) a cAMP/cGMP phosphodiesterase inhibitor such as 3-isobutyl-1-methylxanthine 3-isobutyl-1-methylxanthine?]]] and a (iv) Wnt agonist.

2. The method of claim 1, wherein the Wnt agonist is Wnt3.

3. The method of claim 2, wherein the Wnt3 is at a concentration of about 25-60% v/v; preferably 30-55% for example, 30, 40, 45, 50, 55% v/v.

4. The method of claim 3, wherein IBMX is used at a concentration between about 50 and about 500 μM, preferably between about 75 to 250 μM, more preferably, between 90 and 150 μM, for example about 95, about 100, about 105, or about 110 μM.

5. The method of claim 1, wherein (a) 8-bromocyclic AMP is at concentration between about 50 and about 500 μM, preferably between about 75 to 250 μM, more preferably, between 90 and 150 μM, for example about 95, about 100, about 105, about 110 μM; and/or (b) dexamethasone is used at a concentration between about 25 and 150 nM, preferably between 40 to about 100 nM, and more preferably, between about 45 to 60 nM, for example, about 50 nM or about 55 nM.

6. The method of claim 1, wherein the method further comprises one or more of the following steps prior to culturing the LO in a DD medium:
a. obtaining a lung tissue sample from a subject;
b. obtaining dissociated cells from a lung tissue sample; and
c. culturing lung cells in an lung organoid (LO) formation phase to form a lung organoid (LO) for a period sufficient to generate a LO.

7. The method of claim 6, wherein the LO formation phase comprises culturing cells in an expansion medium comprising one or more components as set out in Table 1, optionally at the concentrations shown in Table 1.

8. The method of claim 7, wherein the expansion medium comprises at least R-spondin, a BMP inhibitor, a TGF-beta inhibitor, FGF and heregulin beta-1.

9. The method of claim 1, wherein the step of culturing the lung cells and/or LO comprises culturing the cells in contact with an exogenous extracellular matrix.

10. The method of claim 1, wherein the LO is a 3D organoid; and/or wherein the AlvO is a 3D organoid.

11. (canceled)

12. A method of generating a AlvO in accordance with claim 1 comprising the steps of:
a. culturing lung cells from a subject in an expansion medium in contact with an extracellular matrix for a period sufficient to generate a 3D LO, for example for at least 2 four weeks and
b. changing the expansion medium to a distal differentiation medium supplemented with a notch inhibitor and culturing the 3D LO in the distal differentiation medium comprising an effective amount of dexamethasone a cyclic AMP (cAMP) agonist such as 8-bromo-cyclic AMP, a cAMP/cGMP phosphodiesterase inhibitor such as 3-isobutyl-1-methylxanthine and a Wnt agonist for a period sufficient to generate an AlvO, for example for at least 5 days, at least 10 days, at least 14 days or at least 16 days.

13. The method of claim 1, wherein: (a) the culture medium is refreshed every other day and/or (b) the organoid or cells are human organoids or human cells.

14. (canceled)

15. An ALvO obtained by claim 1, wherein the AlvO comprises a cell population consisting of at least 40% type I alveolar epithelial (AT1) cells and type II alveolar epithelial (AT2) cells, wherein the AT1 cells are AQP5+ and the AT2 cells are SFTPC+.

16. The AlvO of claim 15, wherein (a) the AlvO has at least 2-fold or at least 3-fold upregulation in AT1 cell markerAQP5 or HOPX and/or AT2 cell markers selected from the group consisting of SFTPA, SFTPB, SFTPC, and SFTPD compared to the LO from which they are obtained; and/or (b) the AlvO further comprises a test virus.

17. The AlvO according to claim 1, wherein the AlvO in suspension culture exhibit an apical-out polarity, as demonstrated by the expression of AQP5 and HTII-280.

18. The AlvO according to claim 16, wherein gene expression is assessed using quantitative PCR of mRNA transcripts normalized with GAPDH.

19. (canceled)

20. A method for contacting a virus in a AlvO, comprising:
a. generating an AlvO in accordance with claim 1; and
b. infecting the AlvO with the test virus, optionally, wherein the test virus is an influenza virus or coronavirus.

21. The method of claim 20, wherein: (a) the infecting step further comprising incubating for at least 30 minutes, at least 60 minutes, at least 90 minutes or at least 120 minutes; and/or (b) the incubating step is performed at about 37° C.

22. (canceled)

23. A method for predicting infectivity of a test virus to humans, comprising:
a. generating a human AlvO in accordance with claim 1;
b. contacting the human AlvO with the test virus;
c. testing the viral titre after a period sufficient to allow viral propagation;
d. optionally comparing the viral titre to a control virus, herein, optionally, the test virus is influenza virus or coronavirus.

24. The method of claim 23, wherein: (a) testing the viral titre involves detecting a change in viral titre; (b) an increase in viral titre is indicative of likely infectivity of the influenza virus to humans and/or wherein a greater increase over a shorter period is correlated with a higher degree of infectivity; and optionally, wherein the increase in viral titre is at least 1 log 10 units, at least 1 log 10 units, or at least 2 log 10 units within 24 hours.

25. (canceled)

26. (canceled)
